(19) United States
(12) Patent Application Publication
(10) Pub. No.: US 2025/0166775 A1
(43) Pub. Date: May 22, 2025

(54) INTERROGATABLE DELIVERY DEVICE AND ASSOCIATED STATUS MODULE

(71) Applicant: NOBLE INTERNATIONAL, LLC, Orlando, FL (US)

(72) Inventors: Jeffery A. LETTMAN, Orlando, FL (US); Randy CARDOZA, Winter Garden, FL (US); Chris EVANS, Rehoboth Beach, DE (US)

(21) Appl. No.: 18/832,988

(22) PCT Filed: Jan. 31, 2023
(86) PCT No.: PCT/US23/11950

§ 371 Date: Jul. 25, 2024

Related U.S. Application Data
(60) Provisional application No. 63/305,196, filed on Jan. 31, 2022.

(51) Int. Cl. G16H 20/17; G16H 40/67

(52) U.S. Cl. CPC G16H 20/17; G16H 40/67

(57) ABSTRACT

An apparatus, including: at least one delivery device (200), each delivery device configured to be either a medicament delivery device or a medicament delivery device trainer, wherein each delivery device includes: a housing (202); a movable component (204) configured to dispense or to simulate dispensing a medicament; and an unpowered electrical circuit (210) having a first lead (212) that is externally accessible, a second lead (216) that is externally accessible, and an electrical loop (218) between the first lead and the second lead and comprising a switch (220). A change in a state of the delivery device either toggles the switch between a closed position and an open position or changes an electrical property of the switch.

### FIELD OF THE INVENTION

The invention relates to medicament delivery devices and trainers which can be interrogated by a status module to determine a state of the delivery device.

### BACKGROUND OF THE INVENTION

Studies of patients who are prescribed self-delivered treatment show that adherence to systemic treatments could be improved. Medication non-adherence is associated with disease flares, increased disability, and increased costs. Median adherence in clinical studies as tracked using self-reported metrics has been shown to be grossly overestimated (17%). This demonstrates the need for training as well as objective monitoring to obtain an accurate picture of adherence.

Current monitoring systems provide some help with the above, but there is room in the art for improvement.

### BRIEF DESCRIPTION OF THE DRAWINGS

The invention is explained in the following description in view of the drawings that show:

FIGS. 1 to 4 are block diagrams representing example embodiments of a delivery device with example embodiments of delivery device interfaces and example embodiments of a status module.

FIG. 5 to FIG. 7 are block diagrams of an example embodiment of a status module with example embodiments of delivery device interfaces.

FIG. 8 is a block diagram of an example embodiment of a status module with an example embodiment of an expanded status module standard interface.

FIG. 9 to FIG. 11 are block diagrams of example embodiments of status module standard interfaces interfacing with example embodiments of delivery device interfaces.

FIG. 12 to FIG. 13 are block diagrams of example embodiments of alignment features used to align the status module standard interface with the delivery device interface.

FIG. 14A to FIG. 14B are schematic representations of an example embodiment of a delivery device showing elements of an unpowered electrical circuit in an open state and a closed state respectively.

FIG. 15A to FIG. 15B are schematic representations of an example embodiment of a delivery device without and with the status module respectively.

FIG. 16A to FIG. 16C are schematic representations showing an alternate example embodiment of a delivery device with the movable component in an unactivated position and with an alternate example embodiment of a status module attached.

FIG. 17A to FIG. 17C are schematic representations showing the alternate example embodiment of the delivery device of FIG. 16A to FIG. 16C with the movable component in an activated position and with the status module of FIG. 16A to FIG. 16C attached.

FIG. 18 is a schematic representation of the delivery device of FIG. 16A to FIG. 16C with the movable component in the unactivated position and with the status module of FIG. 16A to FIG. 16C detached.

### DETAILED DESCRIPTION OF THE INVENTION

The present inventors have developed unique and innovative interrogatable medicament delivery devices and trainers. In an example embodiment, a delivery device includes one or more passive/unpowered electrical circuits, each with a respective switch. A position or electrical property of the switch is associated with a state of the delivery device such as whether an injection or simulated injection has occurred, inter alia. A discrete status module (a.k.a. an interrogation module) can be put into power and/or data communication with the delivery device to provide energy to the unpowered electrical circuit(s) by securing the status module to the delivery device or by simply holding the status module against the delivery device. How the circuit(s) respond to the energy supplied by the status module allows the status module to assess the state of the circuit. The state of the circuit, in turn, indicates the state of the delivery device. The status module can then report this information to a discrete reader via data communication (wireless e.g., Wi-Fi, Bluetooth®, Bluetooth® broadcasting, and/or wired). The information can be used to assess a user's adherence to a treatment regimen and/or to help reduce usage errors attributable to use of a training device.

Unpowered electrical circuits can be as simple as an electrical pathway with some sort of switch. As a result, the unpowered electrical circuits can be incorporated into existing delivery devices relatively easily and inexpensively. For example, each unpowered electrical circuit type may report an associated status. A first unpowered electrical circuit type may report whether an injection has been initiated. A second may report whether an injection has been completed. A third type may report whether a temperature of the delivery device or a substance stored therein has exceeded a threshold temperature during storage. A fourth type may report a current temperature of the delivery device or a substance stored therein. These types are only examples and are not meant to be limiting. Unpowered electrical circuits may report on/be associated with other states than these example states. There may be any number of unpowered electrical circuits that report on any combination of the states in various delivery devices.

A status module may include a status model standard interface configured to interact with an interface on the delivery device. By way of a non-limiting example, lead one and lead two in the status model standard interface may be configured to power/interrogate the first type of unpowered electrical circuit in the delivery device to determine the associated status. Leads three and four may be configured to power/interrogate the second type of unpowered electrical circuit in the delivery device to determine the associated status etc. These leads in the status model standard interface form a standard pattern.

Each delivery device may include a respective delivery device interface that is configured to interact with the status model standard interface. For example, in every delivery device interface, the leads that are associated with the first unpowered electrical circuit type will be positioned to interface with lead one and lead two in the status model standard interface. This is because lead one and lead two of the status model standard interface are configured to power the first type of unpowered electrical circuit. Likewise, in every delivery device interface, the leads that are associated with the second type of unpowered electrical circuit will be positioned to interface with lead three and lead four in the status model standard interface etc. Stated another way, each set of leads in the status model standard interface expects to power a particular type of unpowered electrical circuit to determine the status associated with that type of circuit. As such, every delivery device interface will be configured to position the various leads of the respective types of unpowered electrical circuits where the status model standard interface expects them to be.

A delivery device interface may include leads/contacts arranged in a delivery device pattern that is a mirror image of the standard pattern of the status model standard interface, similar to the arrangement of halves of a conventional wiring harness. Alternately, a delivery device interface may include leads/contacts arranged a delivery device pattern that is a mirror image of a portion of the standard pattern of the status monitor standard interface. In the latter, the standard pattern of the status model standard interface and the delivery device pattern may be configured to fit together in only one way to ensure the proper electrical connections are made between the status module standard interface and the delivery device interface. Stated another way, the delivery device pattern can be any of a variety of delivery device patterns, each of which is configured to interact with the standard pattern of the status module standard interface in only one/correct way. This approach allows one status module to be used to interrogate/query a variety of delivery devices. In addition to the delivery device pattern being the same or different, the different delivery devices themselves can be of the same type (structural configuration and operation) or of different types.

This arrangement allows the unpowered electrical circuit(s) of each delivery device to be uniquely configured inside the delivery device so long as the leads of the unpowered electrical circuit(s) types take positions in the delivery device interface that are expected by the status model standard interface. Moreover, this allows the delivery device interface to have a pattern that is different than the status module standard interface, which provides design flexibility should the need arise.

The status module standard interface may also include more leads than are necessary for the existing number of unique types of unpowered electrical circuits being used in the existing delivery devices. Having extra leads available to connect to the delivery devices allows for expansion of types of unpowered electrical circuits and associated states that may be interrogated should the need arise. For example, if the existing delivery devices incorporate four unique types of unpowered electrical circuits, a status module standard interface having leads for five types of unpowered electrical circuits would allow for a fifth unique type of unpowered electrical circuit to be incorporated into future delivery devices within the existing footprint of the status module standard interface. Further, the status module standard interface may be designed so that its pattern can be expanded in a way that the expanded status module standard interface pattern is fully backwards compatible but adds new leads for future additional unique types of unpowered electrical circuits.

The term “delivery device” includes but is not limited to a training medicament delivery device or an actual medicament delivery device. This includes an injection device such as an auto-injector and/or trainer, an inhaler device and/or respiratory trainer, a bolus injector, a nasal inhaler device, a needleless injector, a transdermal patch, an ampoule, a medicine container, a medicine package, a vial, a metered-dose inhaler, a dry powder inhaler, a prefilled syringe, among other medicament devices known to one of ordinary skill in the art.

Such a status module could be used to interrogate one or more delivery devices of one or more individuals several times over a period of time. Information gathered by the interrogations can be used, for example, to assess adherence to substance delivery regimes. This could be valuable information for a healthcare professional who is training a patient remotely (e.g., via a teleconference or the like). The data would allow the healthcare professional to know what step the patient is on in real time and communicate any misuse issues that might be hard to see through a video conference.

Locating the more complex aspects of the arrangement in the status module, as opposed to locating the more complex aspects in each delivery device, keeps the cost of the delivery device down, which keeps overall costs of the arrangement down.

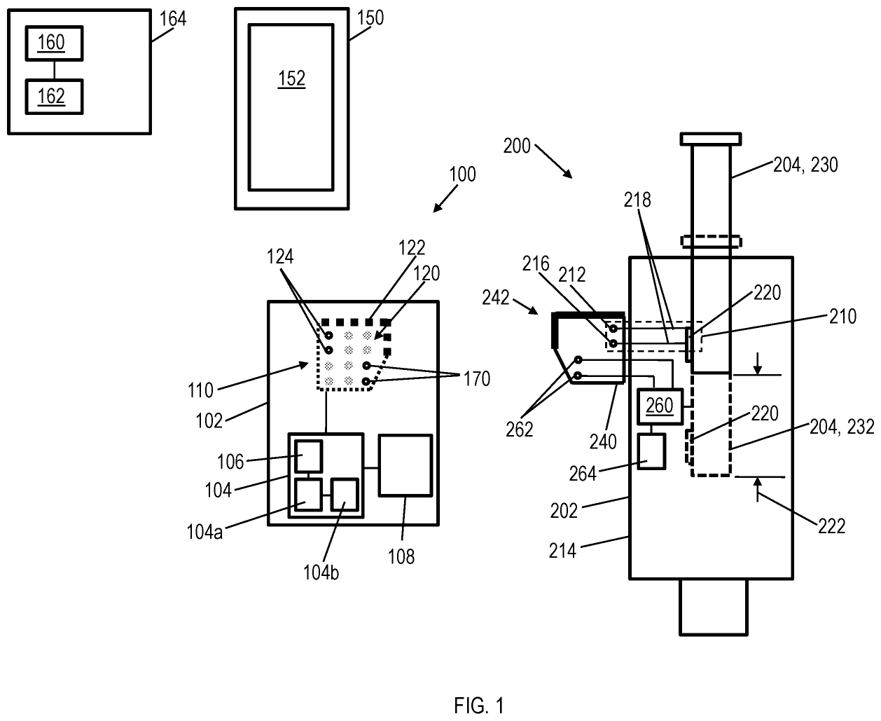

FIG. 1 is a block diagram of an example embodiment of a status module 100 and a delivery device 200. The status module 100 includes a status module housing 102, a status module power source 104, a status module controller 106, a status module transmitter/receiver 108, and a status module standard interface 110 having standard module interface leads 120. The status module power source 104, the status module controller 106, and the status module transmitter/receiver 108 are all in electrical and/or data communication with each other.

The status module power source 104 may include a dedicated status module battery 104a that can power the status module 100. Alternately, or in addition, the status module power source 104 may include a status module energy harvesting device 104b, which may be any type of energy harvesting device. In an example embodiment, the status module energy harvesting device 104b is a field energy harvesting device configured to receive energy from a field. Such a field energy harvesting device can harvest energy generated by a near field communication (NFC) system of, for example, a cell phone. When the field energy of the NFC device is available to the status module 100, the field energy harvesting device can harvest this energy to power the status module 100 and/or to charge the status module battery 104a. Alternately, the status module energy harvesting device 104b can be, for example, a mechanical energy harvesting device configured to harvest energy from motion of the status module. When the status module 100 is moved, the mechanical energy harvesting device can harvest this energy to power the status module 100 and/or to charge the status module battery 104a. Such status module energy harvesting devices offer the flexibility to power the status module 100 when the status module 100 doesn't have a status module battery 104a, when the status module battery 104a is weak or dead, and/or when the status module 100 is not otherwise powered by, for example, the delivery device 200.

The status module 100 is configured to establish data communication (wired and/or wireless communication) with a discrete reader 150 having a graphic user interface 152. Example readers include smart devices (e.g., a smartphone, tablet, smartwatch, smart glasses or other personal electronic device). Data communication herein, such as that between the status module 100 and the reader 150, may be via Wi-Fi, Bluetooth®, Bluetooth® broadcasting, and the like.

Bluetooth® broadcasting, as opposed to standard BT communication, is an aspect of the Bluetooth® Low Energy (BLE) standard that does not requiring a pairing step. For applications where the amount of data exchanged is sufficiently small, it can be transmitted as part of the 31 bytes available with the advertising packet. This offers a number of advantages. 1) There is no need for the user to pair the status module 100 device with the reader 150 (e.g., smartphone). Communication is possible as soon as the two are within range of each other. 2) The technical requirements of the Bluetooth® microcontroller are much lower, enabling the use of cheaper, “disposable”, devices. 3) The status module 100 does not need to receive signals from the reader 150. Since the communication is only one-direction, the power-requirements in operation can be much lower. 4) The power savings enable powering the device with a 1.5V coin cell battery rather than 3V batteries (as is typical for connected devices). 1.5V batteries are significantly lower cost, and smaller, than 3V batteries.

A controller 160 may include one or more algorithms in data communication with a database 162 and be configured to gather data from the status module 100 and/or the reader 150. The algorithms may be configured to use the data gathered by the status module 100 to identify errors (e.g., usage and/or adherence errors) and corrective actions from the database 162 to generate corrective action recommendations for the user that are designed to correct the identified errors. In this example embodiment, the controller 160 and/or the database 162 are be disposed on a server 164 that is remotely accessible by the reader and/or the status module 100. Alternately, the controller 160 and/or the database 162 disposed on or among any or all of the status module 100, the reader 150, and the remotely accessible server(s) 164.

The controller 160 and/or the reader 150 may be configured to provide training/instructions (audio, visual, or audio/visual together) to the user. The training may provide step-by-step instructions and may be done in real time. In addition, real time training may use information transmitted from the status module 100 during a real or simulated injection to determine adherence with the step-by-step training. In an example embodiment the process may include the following steps. 1) The user opens the associated training app on the reader 150 (e.g., smart phone). The app is listening for communication/broadcasts from the status module 100 once opened (no pairing step is required when using Bluetooth® broadcasting). 2) The app instructs the user, with visual and/or spoken prompts to remove a cap. When the cap is removed, the status module 100 broadcasts the updated status of the delivery device 200, and the app advances to display the next set of instructions. 3) Once the real or simulated injection occurs, the app is alerted to the start of the real or simulated injection process. The app displays a countdown to let the user know how long they should hold the delivery device 200 in place. The status module 100 continues to provide the current status of the delivery device 200 during this countdown. If the status doesn't change, the real or simulated injection was completed correctly, if the status changes because the delivery device 200 was prematurely removed from the injection site, that error is communicated to the user.

The controller 160 and/or the reader 150 may be configured to provide links to additional training materials (e.g., videos, instructions for use, etc.) when an error occurs, at the request of the user, or any other time deemed suitable.

The controller 160 and/or the reader 150 may be configured to enable an audio and/or a video conference with a healthcare professional regarding onboarding, training, adherence (e.g., as indicated by the delivery device 200) and the like.

The controller 160 and/or the reader 150 may be configured to understand when a user is looking for additional training.

In addition, the controller 160 and/or the reader 150 may be configured to assist in onboarding a new patient by describing the operation of the delivery device 200 and/or use of the reader 150 etc.

The delivery device 200 includes a delivery device housing 202, a movable component 204 configured to dispense or to simulate dispensing a medicament, and an unpowered electrical circuit 210 including a first lead 212 that is externally accessible on or about a surface 214 of the delivery device housing 202, a second lead 216 that is externally accessible on or about the surface 214 of the delivery device housing 202, an electrical loop 218, and a switch 220 between the first lead 212 and the second lead 216. The first lead 212 and the second lead 216 are bolded relative to other standard interface leads 120 in the figures to highlight their position. In this example embodiment, the switch 220 may be a conductive material that is secured to and/or integrated into the movable component 204.

In this example embodiment, the movable component 204 represents a plunger associated with a dispensing or a simulated dispensing of a medicament. The movable component 204 shown solid represents an unactivated/undispensed position 230 of the movable component 204. The movable component 204 shown in dashed lines represents an activated/dispensed position 232 of the movable component 204 after the movable component 204 has moved through a dispensing stroke 222. In the unactivated/undispensed position 230, the switch 220 closes electrical loop 218. This closes the unpowered electrical circuit 210. Moving from the unactivated/undispensed position 232, moves the switch 220 out of the electrical loop 218. This opens the unpowered electrical circuit 210. Hence, a position of the switch 220 is associated with a position of the movable component 204, and the change in state incudes a select movement of the movable component 204, where the select movement includes moving the movable component 204 from the undispensed position 230. The amount of movement it takes to open the unpowered electrical circuit 210 can be adjusted by, for example, adjusting a length and/or position of the switch 220 so that all it takes is any movement to open the unpowered electrical circuit 210, or so that it takes a minimum amount of movement to open the unpowered electrical circuit 210. In this example embodiment and for discussion purposes, the unpowered electrical circuit 210 may be referred to as a type 1 unpowered electrical circuit because it indicates whether an injection has been initiated. Although types of circuits are discussed herein, this is for explanatory reasons and is not intended to limit the disclosure to these types or these exact configurations.

The status module standard interface 110 includes several status module standard interface leads 120 arranged in a standard pattern and surrounded by an alignment feature 122. The alignment feature 122 is used in conjunction with an associated alignment feature 240 on the delivery device 200 to ensure proper alignment between the status module standard interface 110 and a delivery device interface 242 in which the first lead 212 and the second lead 216 are disposed. (The patterns are shown as mirror to each other because the views show the interfacing/abutting sides of the interfaces.) Included in the several status module standard interface leads 120 are two standard module interface unpowered electrical circuit leads 124. The two standard module interface unpowered electrical circuit leads 124 are bolded relative to other standard interface leads 120 to highlight their position.

When the status module standard interface 110 interfaces with the delivery device interface 242, the two standard module interface unpowered electrical circuit leads 124 interact/interface with the first lead 212 and the second lead 216. The two standard module interface unpowered electrical circuit leads 124 may be referred to as two type 1 standard module interface unpowered electrical circuit leads 124 because they power the type 1 unpowered electrical circuit 210 in this example embodiment.

In an example embodiment, one of the two standard module interface unpowered electrical circuit leads 124 provides power to the unpowered electrical circuit 210. If the other of the two standard module interface unpowered electrical circuit leads 124 receives the power, the unpowered electrical circuit 210 is necessarily closed by the switch 220. The status module 100 uses this information to infer that the movable component 204 is in the undispensed position 230. Alternately, if the other of the two standard module interface unpowered electrical circuit leads 124 does not receive the power, the unpowered electrical circuit 210 is necessarily open. The status module 100 uses this information to infer that the movable component 204 has been moved from the undispensed position 230. These inferences can be used to determine/infer adherence to a treatment regimen and/or can be used as part of a training program designed to reduce usage errors.

Every delivery device 200 that has a type 1 unpowered electrical circuit 210 (e.g., whether an injection has been initiated) will position the first lead 212 and the second lead 216 in the respective delivery device interface 240 so that the first lead 212 and the second lead 216 correspond with the two type 1 standard module interface unpowered electrical circuit leads 124 when the delivery device interface 242 interfaces with the status module standard interface 110. This is possible because the delivery device interface 242 is configured to be able to interact with the status module standard interface 110 in only one way. The same applies for each of the types of unpowered electric circuits in the various example embodiments disclosed herein.

In an example embodiment, the delivery device 200 includes a delivery device energy harvesting device 260, which can be any type of energy harvesting device. In an example embodiment, the delivery device energy harvesting device 260 is a mechanical energy harvesting device. The delivery device mechanical energy harvesting device can be associated with the movable component 204. In such a configuration, when the movable component 204 (e.g., the plunger) is depressed, or reset in the case of a resettable movable component, the delivery device energy harvesting device 260 can harvest energy which can be used to supplement or fully power the status module 100.

In such an example embodiment, the delivery device energy harvesting device 260 can deliver energy to power supply leads 262 in the delivery device interface 242. The power supply leads 262 will contact and deliver the energy to power load leads 170 when the status module standard interface 110 interfaces with the delivery device interface 242. The power load leads 170 can, in turn, deliver the energy to the status module power source 104 to supplement the status module power source 104 (e.g., to supplement a dedicated battery). Alternately, the power load leads 170 can deliver the energy directly throughout the status module 100 as needed. The delivery device 200 optionally includes a delivery device energy harvesting device battery 264 configured to store any energy the delivery device energy harvesting device 260 harvests. The delivery device energy harvesting device battery 264 is further configured to deliver the energy to the power supply leads 262 when the status module standard interface 110 interfaces with the delivery device interface 242, regardless of whether the delivery device energy harvesting device 260 is actively harvesting energy or not.

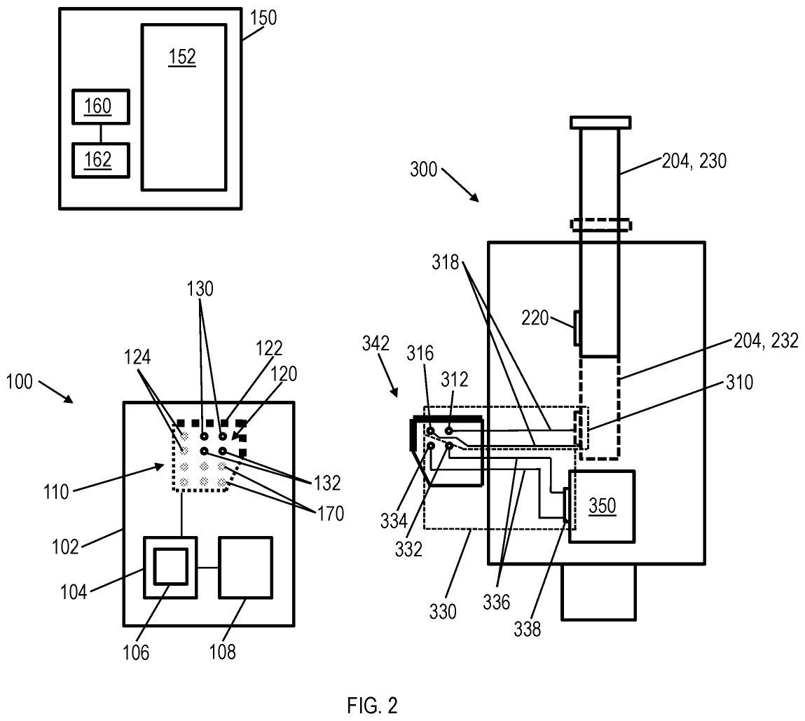

FIG. 2 is a block diagram of an example embodiment of a status module 100 and an example embodiment of a delivery device 300. The delivery device 300 is similar to the delivery device 200 but includes differences as discussed and shown. This example embodiment of the delivery device 300 includes an unpowered electrical circuit 310 that includes a third lead 312, a fourth lead 316, and an unpowered electrical loop 318. In this example embodiment, moving the movable component 204 into the activated/dispensed position 232 moves the switch 220 into the unpowered electrical loop 318. This closes the unpowered electrical circuit 310. Hence, the change in state includes moving the movable component 204 into the activated/dispensed position 232. In this example embodiment, the unpowered electrical circuit 310 may be referred to as a type 2 unpowered electrical circuit because it indicates whether an injection has been completed. This status can be used, for example, in conjunction with a type 1 status to infer if an injection which is initiated (type 1) is subsequently completed (type 2).

Included in the several status module standard interface leads 120 are two standard module interface unpowered electrical circuit leads 130. The two standard module interface unpowered electrical circuit leads 130 are bolded relative to other standard interface leads 120 to highlight their position. When the status module standard interface 110 interfaces with the delivery device interface 342, the two standard module interface unpowered electrical circuit leads 130 interact/interface with the third lead 312 and the fourth lead 316. The two standard module interface unpowered electrical circuit leads 130 may be referred to as two type 2 standard module interface unpowered electrical circuit leads 130 because they power the type 2 unpowered electrical circuit 310 in this example embodiment.

In an example embodiment, one of the two standard module interface unpowered electrical circuit leads 130 provides power to the unpowered electrical circuit 310. If the other of the two standard module interface unpowered electrical circuit leads 130 receives the power, the unpowered electrical circuit 310 is necessarily closed by the switch 220. The status module 100 uses this information to infer that the movable component 204 is in the dispensed position 232. Alternately, if the other of the two standard module interface unpowered electrical circuit leads 130 does not receive the power, the unpowered electrical circuit 310 is necessarily open. The status module 100 uses this information to infer that the movable component 204 has not been moved into the dispensed position 232. These inferences can be used to determine/infer adherence to a treatment regimen and/or can be used as part of a training program designed to reduce usage errors.

This example embodiment of the delivery device 300 further includes an unpowered electrical circuit 330 that includes a fifth lead 332, a sixth lead 334, an unpowered electrical loop 336, and a temperature responsive switch 338. The temperature responsive switch 338 is in thermal communication with a substance 350 in the delivery device 300. The substance 350 may be a medicament, a medicament substitute, or any other component of the delivery device 300.

In one example embodiment, the temperature responsive switch 338 may be configured to permanently change an electrical property if a temperature of the substance 350 warms to at least the predetermined temperature. For example, if the substance 350 is meant to be stored below a certain temperature, the temperature responsive switch 338 can be configured to change its electrical property if the substance 350 exceeds that temperature during storage. The electrical property can then be interrogated by the status module 100. For example, the temperature responsive switch 338 may be configured to change from nonconductive to conductive once the threshold temperature is exceeded, or vice versa. In this example embodiment, the unpowered electrical circuit 330 may be referred to as a type 3 unpowered electrical circuit because it indicates whether a temperature of the substance 350 has been exceeded.

The status module 100 may include standard module interface unpowered electrical circuit leads 132 configured to interface with the fifth lead 332 and the sixth lead 334. The two standard module interface unpowered electrical circuit leads 132 may be referred to as two type 3 standard module interface unpowered electrical circuit leads 132 because they power the type 3 unpowered electrical circuit 330 in this example embodiment. The status module 100 can interrogate the status of the temperature responsive switch 338 in a manner similar to that described above to determine if the temperature of the substance 350 warmed above a threshold temperature. These inferences can be used to determine/infer adherence to regimen requirements such as storage conditions and/or can be used as part of a training program designed to reduce usage errors.

In this example embodiment, the controller 160 and the database 162 are disposed on the reader 150.

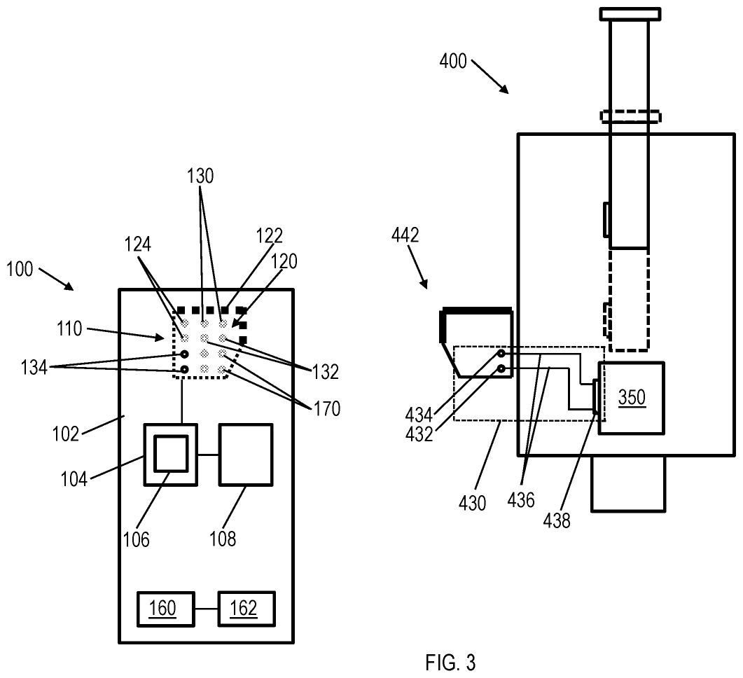

FIG. 3 is a block diagram of an example embodiment of a status module 100 and an example embodiment of a delivery device 400. The delivery device 300 is similar to the delivery device 200 but includes differences as discussed and shown.

This example embodiment of the delivery device 400 includes an unpowered electrical circuit 430 that includes a seventh lead 432, an eighth lead 434, an unpowered electrical loop 436, and a temperature responsive switch 438. The temperature responsive switch 438 is in thermal communication with a substance 350 in the delivery device 400.

In one example embodiment, the temperature responsive switch 438 may be configured to include an electrical property that can be used to infer a current temperature of the substance 350. For example, the electrical property may be a resistance associated with the temperature responsive switch 438 that changes with temperature. In this example embodiment, the unpowered electrical circuit 430 may be referred to as a type 4 unpowered electrical circuit because it indicates a current temperature of the substance 350.

The status module 100 may include standard module interface unpowered electrical circuit leads 134 configured to interface with the seventh lead 432 and the eighth lead 434. The two standard module interface unpowered electrical circuit leads 134 may be referred to as two type 4 standard module interface unpowered electrical circuit leads 134 because they power the type 4 unpowered electrical circuit 430 in this example embodiment. The status module 100 can interrogate the status of the temperature responsive switch 438 by measuring a resistance of the temperature responsive switch 438 to determine the current temperature of the substance 350. This information can then be used to determine an adherence to regimen requirements such as conditions appropriate for dispensing (e.g., a minimum temperature) and/or can be used as part of a training program designed to reduce usage errors.

In this example embodiment, the controller 160 and the database 162 are disposed on the status module 100. In such example embodiment, the controller 160 and the database 162 can be updated respectively as necessary via the data communication with the reader 150 and its data communication with the internet.

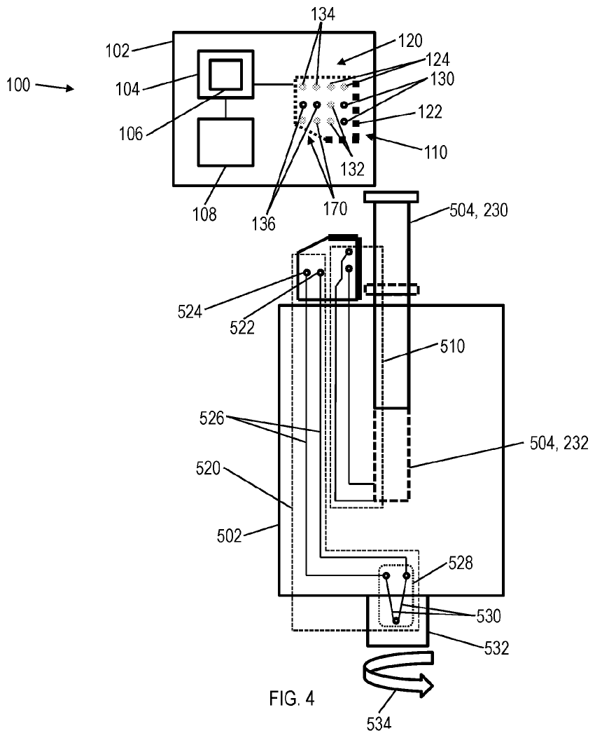

FIG. 4 is a block diagram of an example embodiment of a status module 100 and an example embodiment of a delivery device 500. The delivery device 500 is similar to the delivery device 200 but includes differences as discussed and shown. In this example embodiment, the movable component 504 operates the same as the movable component 204 of FIG. 1. However, in this example embodiment, the movable component 504 is composed either entirely or partly of a conductive material (e.g., metal, conductive plastic). The conductive material can take the place of the switch 220. Hence, the movable component 504 itself closes the (e.g., type 2) unpowered electrical circuit 510 when the movable component 504 is in the dispensed position 232. In the latter example embodiment where the movable component 504 is composed only partly of conductive material, the conductive material is disposed in a part of the movable component 504 that closes the unpowered electrical circuit 510 when the movable component 504 is in the dispensed position 232.

The delivery device 500 further includes an unpowered electric circuit 520 with a ninth lead 522, a tenth lead 524, an electric loop 526, and a switch 528 having frangible links 530. The frangible links 530 may be connected between a stationary part of the delivery device such as a delivery device housing 502 and a movable and/or removable component 532. In an example embodiment, the removable component 532 may be a cap. In an example embodiment, the cap may be removed via a twisting movement 534. The twisting movement 534 would break the frangible links 530 which would change a status of the unpowered electric circuit 520 from closed to open. In this example embodiment, the unpowered electrical circuit 510 may be referred to as a type 5 unpowered electrical circuit because it indicates whether the cap has been removed.

The status module 100 may include standard module interface unpowered electrical circuit leads 136 configured to interface with the ninth lead 522 and the tenth lead 524. The two standard module interface unpowered electrical circuit leads 136 may be referred to as two type 5 standard module interface unpowered electrical circuit leads 136 because they power the type 5 unpowered electrical circuit 520 in this example embodiment. The status module 100 can interrogate the status of the temperature switch 528 to determine the status of the unpowered electrical circuit 520 in a manner disclosed above. From this, the status module 100 can infer if the removable component 532 has been removed. A removal of the removable component 532 may be used as a basis to infer that dispensing or simulated dispensing has occurred, which can be used to determine an adherence to a treatment regimen and/or can be used as part of a training program designed to reduce usage errors.

Any status that can be checked via an interrogatable electrical property is within the scope of the disclosure. Additional example states include whether a needle shield is in a retracted position or an extended position, and the associated change of state is whether the needle shield has moved from the retracted position or into the extended position. Another example state includes whether the needle has been inserted. Further states include any position of any moveable component of the delivery device, with associated changes of state being movement from or into the respective position.

Likewise, any internal configuration of the loops is within the scope as well as any pin patterns, shapes, and locations.

Use of the status module in conjunction with a delivery device allows for the determination of sequence of use as well as the timing associated with this sequence. This information allows the status module to determine not just discrete actions, but proper use of the delivery device. This information about proper use and associated error correction activities can be provided to the user through the reader (e.g., smartphone) in communication with the status module via Bluetooth®, Bluetooth Broadcasting®, and/or Wi-Fi, and the like. Such error correction activities can be generated by an algorithm that considers any or all of the data gathered by the status module, determines errors based on the data, and generates the error correction activities from, for example, a database of known error correction activities. The algorithm and database may be part of a controller that is disposed on or distributed among any or all of the status module, the discrete reader, and remotely accessible servers.

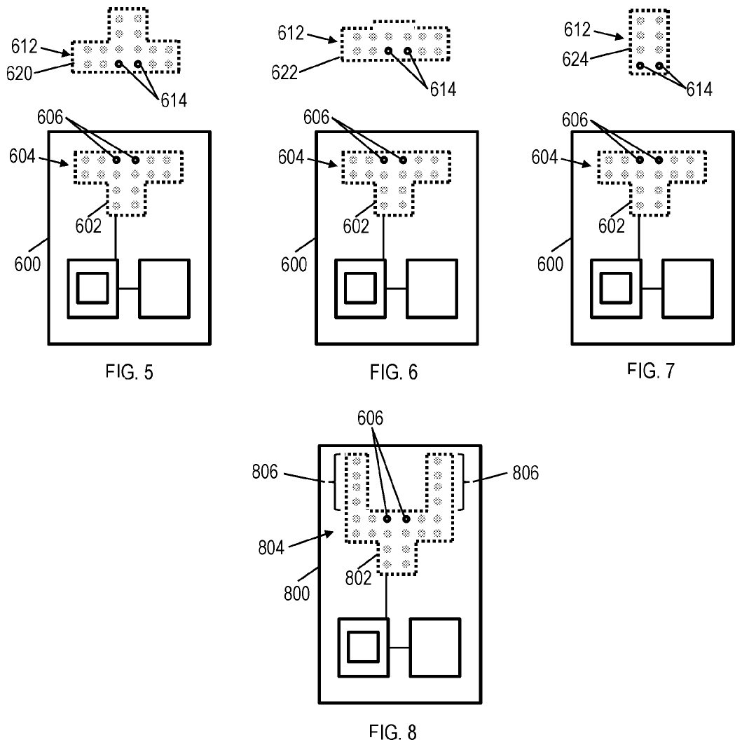

FIG. 5 to FIG. 7 are block diagrams of an example embodiment of a status module 600 with various example embodiments of delivery device interfaces (the delivery devices are not shown). Each delivery device interface has a unique shape, but the unique shape can interact with the status module standard interface 602 in only one way. (The patterns are shown as mirror to each other because the views show the interfacing/abutting sides of the interfaces.)

The status module standard interface 602 includes status module standard interface leads 604 that include two status module standard interface leads 606. The two status module standard interface leads 606 may be associated with a respective type of unpowered electrical circuit (e.g., type 1) in the delivery devices. The delivery device interfaces include delivery device lead 612 that include delivery device leads 614. The two delivery device leads 614 may be associated with a respective type of unpowered electrical circuit (e.g., type 1).

In the example embodiment of FIG. 5, the pattern of leads 612 in the delivery device interface 620 is the same as the pattern of status module standard interface 602. Hence, the interfaces fit together in only one way. This ensures that the two status module standard interface leads 606 interface with the two delivery device leads 614.

In the example embodiment of FIG. 6, the pattern of leads 612 in the delivery device interface 622 is different than (e.g., a subset of) the pattern of status module standard interface 602. However, the pattern of leads 612 can only interface with the status module standard interface leads 604 in one way. Here again, this ensures that the two status module standard interface leads 606 interface with the two delivery device leads 614.

Likewise, in the example embodiment of FIG. 7, the pattern of leads 612 in the delivery device interface 624 is different than (e.g., a subset of) the pattern of status module standard interface 602 and different than in delivery device interface 622. However, the pattern of leads 612 can only interface with the status module standard interface leads 604 in one way, which ensures that the two status module standard interface leads 606 interface with the two delivery device leads 614.

Hence, the status module standard interface 602 can interface with a variety of delivery device interfaces having different lead patterns than the status module standard interface 602 and different lead patterns than each other. This provides great flexibility in how the delivery device interface can be incorporated into various current and future delivery devices.

FIG. 8 is a block diagram of an example embodiment of a status module 800 having an example embodiment of an expanded status module standard interface 802. The expanded status module standard interface 802 includes the same pattern of status module standard interface leads 804 present in the status module standard interface 602 of FIGS. 6-8, plus additional leads 806. The delivery device interfaces 620, 622, and 624 can still only interface with the expanded status module standard interface 802 in one way. This ensures that the two status module standard interface leads 606 still always interface with the two delivery device leads 614. However, the additional leads 806 allow for an expansion of the types of unpowered electrical circuits that can be installed in the delivery devices. The expanded status module standard interface 802 is thereby backwards compatible while providing expansion. Such an expansion can be repeated as necessary.

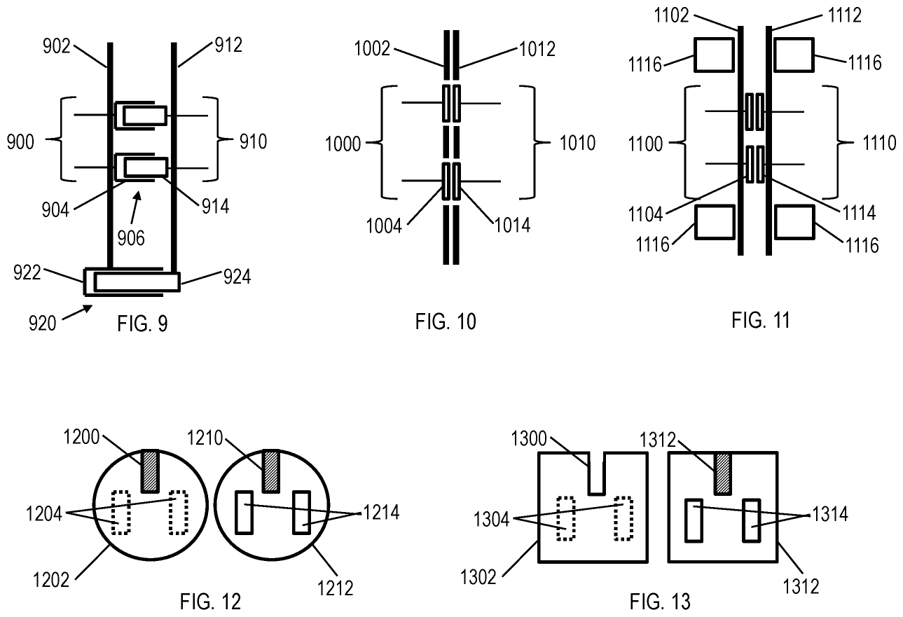

FIG. 9 to FIG. 11 are block diagrams of example embodiments of status module standard interfaces interfacing with various example embodiments of device interfaces.

In the example embodiment of FIG. 9, electrical/data communication is establishes via a mechanical coupling. The status module standard interface 900 of the status module 902 includes a lead that takes the form of a first half 904 of a mechanical coupling 906. The delivery device interface 910 of the delivery device 912 includes a lead that takes the form of a second half 914 of the mechanical coupling 906. The mechanical coupling 906 may be any type of electrical connection known to the artisan that uses an (e.g., reusable) mechanical mating (e.g., pin and socket etc.), and the first half 904 and the second half 914 may be reversed from that shown.

In the example embodiment of FIG. 9, in addition to using a mechanical coupling 906 to establish electrical/data communication, a securing mechanical coupling 920 may be used to physically secure the status module standard interface 900 to the delivery device interface 910. An example of a securing mechanical coupling 920 (e.g., a reusable mechanical coupling) is an electrical harness or the like having a first half 922 and a second half 924 that mechanically interlock to maintain the interface. The securing mechanical coupling 920 may be any type of mechanical connection known to the artisan (sprung connections, friction connections, fasteners, releasable adhesive, hook and loop fasteners etc.) that uses the first half 922 and the second half 924, which may be reversed from that shown. When the status module 902 is mechanically coupled to the delivery device 912, the status module 902 can interrogate the delivery device 912.

In the example embodiment of FIG. 10, electrical/data communication is established via simply abutting flat leads and manually holding the status monitor 1002 in place against the delivery device 1012. The status module standard interface 1000 of the status module 1002 includes a flat lead 1004. The delivery device interface 1010 of the delivery device 1012 includes a flat lead 1014. When the status module 1002 is manually held against the delivery device 1012, the electrical/data communication is established and the status module 1002 can interrogate the delivery device 1012.

In the example embodiment of FIG. 11, the status module standard interface 1100 of the status module 1102 includes a flat lead 1104 and the delivery device interface 1110 of the delivery device 1112 includes a flat lead 1114. Once the status module 1102 is manually brought near the delivery device 1112, magnets 1116 draw the status module 1102 and the delivery device 1112 together and hold them in place. This establishes the electrical/data communication and the status module 1102 can interrogate the delivery device 1112.

FIG. 12 to FIG. 13 are block diagrams of example embodiments of alignment features used to align the status module standard interface with the device interface. These may be use alone or in conjunction with the features of FIG. 9 to FIG. 11.

In the example embodiment of FIG. 12, a visual indicator 1200 associated with the status module standard interface 1202 may be aligned with a visual indicator 1210 associated with the delivery device interface 1212 to ensure status module standard interface leads 1204 properly align with delivery device leads 1214.

In the example embodiment of FIG. 13, a recess 1300 associated with the status module standard interface 1302 may be keyed with a key 1310 associated with the delivery device interface 1312 to ensure status module standard interface leads 1304 properly align with delivery device leads 1314.

In another example embodiment, a type of circuit may be used to confirm alignment or indicate improper alignment.

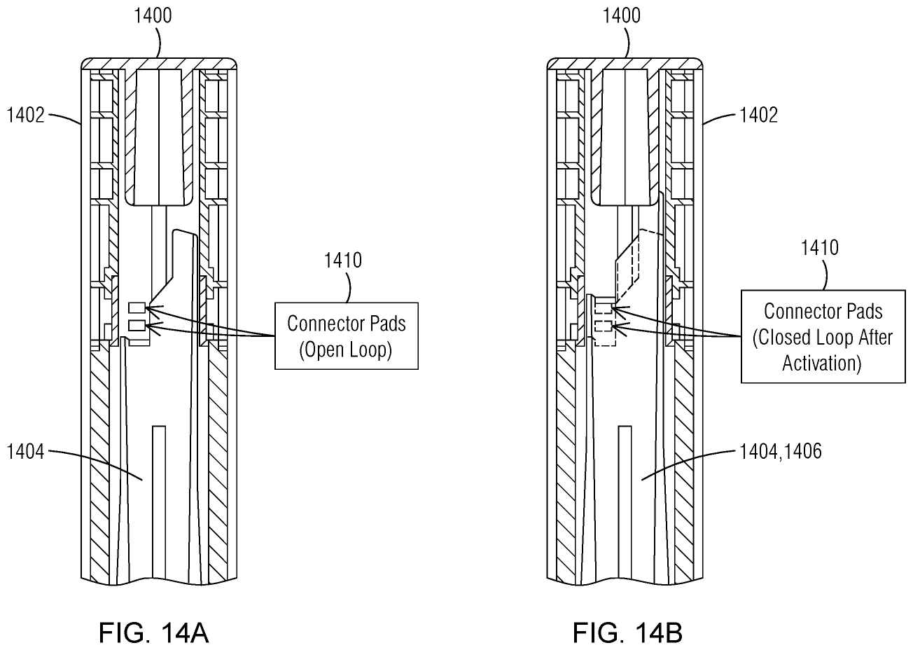

FIG. 14A to FIG. 14B are schematic representations of an example embodiment of a delivery device 1400 showing elements of an unpowered electrical circuit in an open state and a closed state respectively. The delivery device includes a delivery device housing 1402 and a movable component 1404 movable from outside of the dispensed/activated position (FIG. 14A) into the dispensed/activated position 1406 (FIG. 14B). Connector pads 1410 of the (type 2) unpowered electrical circuit (not shown) are exposed when the movable component is not in the dispensed/activated position 1406. When the connector pads 1410 are exposed like shown in FIG. 14A, the status monitor can register that the unpowered electrical circuit is open and infer that the delivery device 1400 has not been activated.

In this example embodiment, the movable component 1404 is composed of a conductive material. Moving the movable component 1404 into the dispensed/activated position 1406 (FIG. 14B), causes the conductive material of the movable component 1404 to contact the connector pads 1410 of the unpowered electrical circuit (not shown). When the connector pads 1410 are in contact with the conductive material of the movable component 1404 as in FIG. 14B, the status monitor can register that the unpowered electrical circuit is closed and infer the delivery device 1400 has been activated.

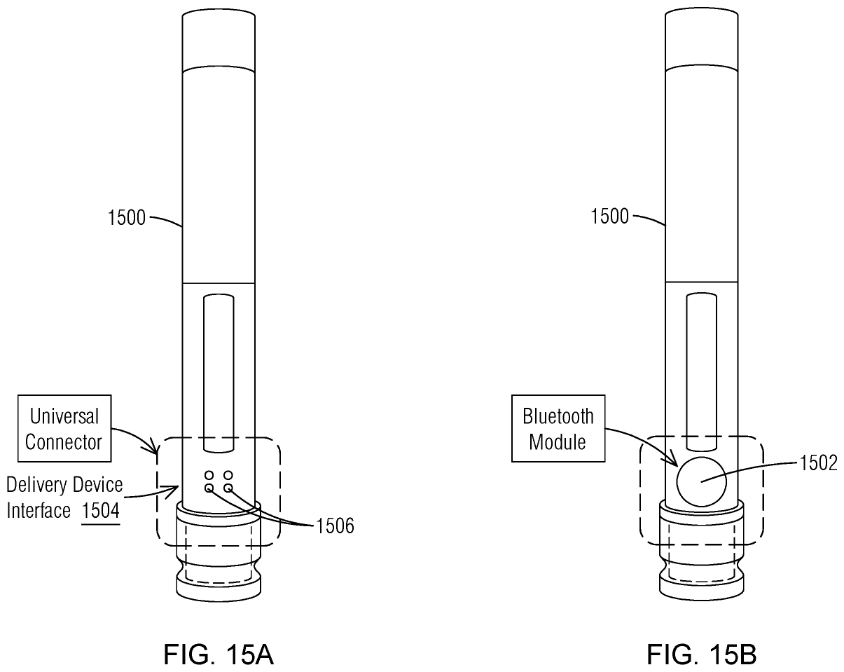

FIG. 15A to FIG. 15B are schematic representations of an example embodiment of a delivery device 1500 without and with the status module 1502 respectively. In FIG. 15A, the delivery device interface 1504 and delivery device leads 1506 are visible. The status module in FIG. 15B may be manually held in place against the delivery device 1500, or magnetically held in place etc., as described above.

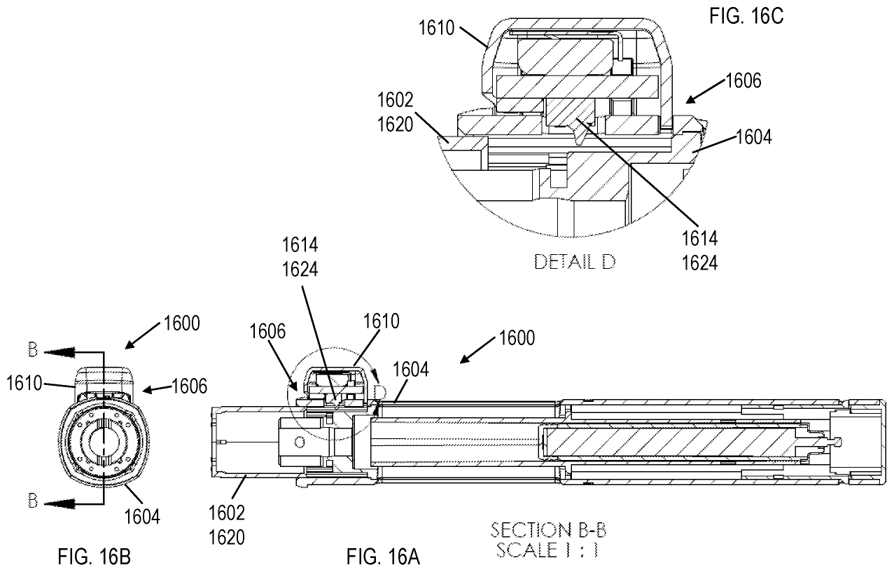

FIG. 16A to FIG. 18 are schematic representations showing an alternate example embodiment of a delivery device 1600 with an alternate example embodiment of a status module 1610. In this example embodiment, the circuit and switch are moved to the status module 1610.

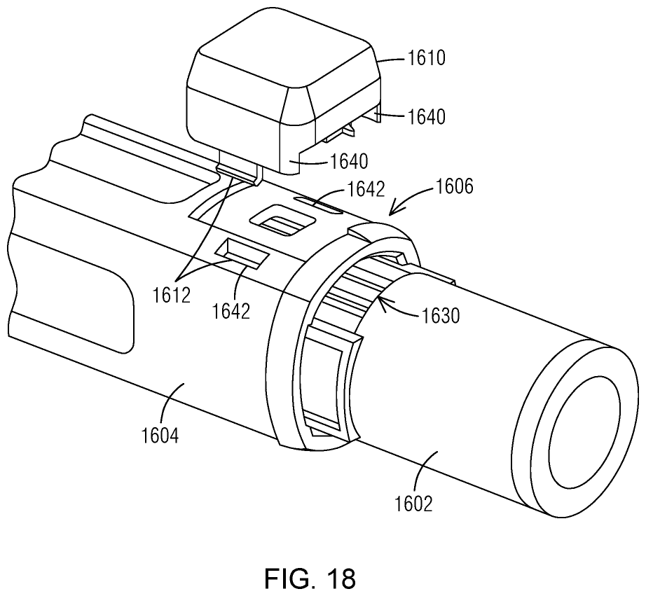

FIG. 16A to FIG. 16C show the delivery device 1600 with a movable component 1602 in the unactivated position. In this example embodiment, the movable component 1602 is a needle shield. However, any component that moves as part of an injection or simulated injection can be suitable. The delivery device 1600 further includes a housing 1604 and a housing interface 1606. A status module 1610 is associated with (e.g., releasably/reusably secured to) the housing 1604 via a mechanical coupling 1612 (see FIG. 18) therebetween and includes a switch 1614.

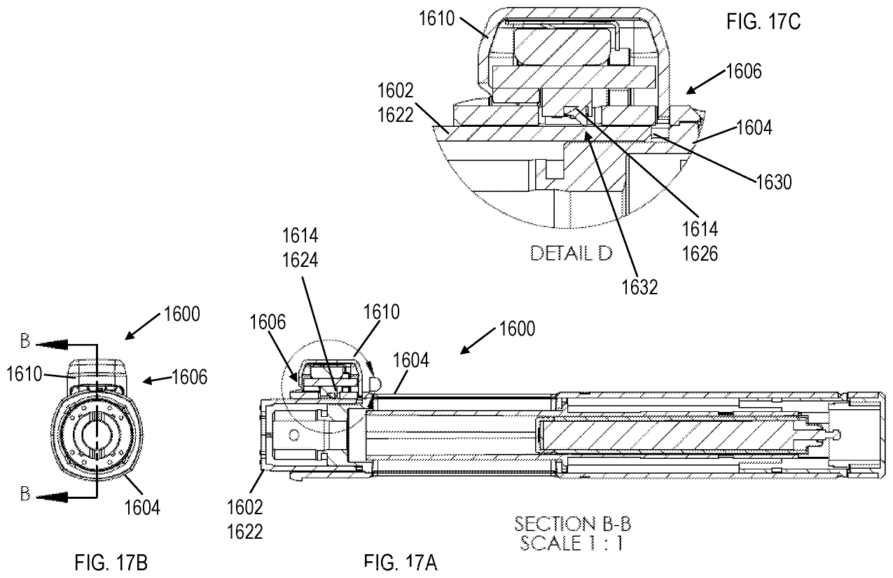

FIG. 17A to FIG. 17C show the delivery device 1600 with the movable component 1602 in the activated position.

As can best be seen FIG. 16C, when the status module 1610 is associated with the housing interface 1606 and when the movable component 1602 is in the unactivated position 1620, the switch 1614 is in a switch first position 1624 (e.g., a switch unactivated position). As can best be seen FIG. 17C, when the status module 1610 is associated with the housing interface 1606 and when the movable component 1602 is in the activated position 1622, the switch 1614 is in a switch second position 1626 (e.g., a switch activated position). In an example embodiment, the switch first position 1624 can be a position in which the switch 1614 opens a circuit (e.g., in the status module 1610) (not shown) or a position in which the switch 1614 closes a circuit. Consequently, the position/state of the movable component 1602 will control the position/state of the switch 1614 and the position/state of the switch 1614 will control the state of the circuit, so the state of the circuit will indicate the state/position of the movable component 1602. Since the state/position of the movable component 1602 indicates whether an injection or a simulated injection has been initiated and/or completed, the state of the circuit will indicate whether the injection or simulated injection has been initiated and/or completed.

In the example embodiment shown, the movable component 1602 moves under the switch 1614 as the movable component 1602 moves toward the activated position 1622. This moves the switch 1614 from the switch first position 1624 to the switch second position 1626. While not explicitly shown, the opposite is equally possible. For example, when the movable component 1602 is in the unactivated position 1620, there could be a portion of the movable component 1602 that is disposed under the switch 1614 and that holds the switch 1614 in the switch second position 1626. In such an embodiment, when the switch 1614 moves toward the activated position 1622, the portion of the movable component 1602 that was under the switch 1614 could move out from thereunder, thereby causing the switch 1614 to drop from the switch second position 1626 to the switch first position 1624.

As can be seen in FIG. 17C, enroute to the activated position 1622, a leading edge 1630 of the movable component 1602 will contact and activate the switch 1614 by moving the switch 1614 from the switch first position 1624 to the switch second position 1626 when the leading edge 1630 reaches a threshold position 1632 (e.g., a second threshold position). This causes the state of the switch 1614 to change before the movable component 1602 reaches the activated position 1622. The activated position 1622 may thereby be considered a fully activated position. The movable component 1602 may be configured such that a sufficient completion of a dispensing operation or a simulated dispensing operation occurs when the movable component 1602 reaches the threshold position 1632. In such an example embodiment, since a sufficient completion of a dispensing operation or a simulated dispensing operation has occurred, it is acceptable for the state of the switch 1614 to be changed. The threshold position 1632 may be separated from the activated position 1622 like shown. The amount of separation may be selected based on any suitable criteria. In an example embodiment, the threshold position 1632 and the fully activated position 1622 may be the same position.

In another example embodiment, when the movable component 1602 moves from the unactivated position 1620, an edge/feature of the movable component 1602 will move beyond a threshold position (e.g., a first threshold position) and cause the switch 1614 to change state. This threshold position may be at a configured so that the switch 1614 changes state once the movable component moves a select distance from the unactivated position 1620. Alternately, the movable component 1602 may be configured to change the state of the switch 1614 with any movement of the movable component 1602 away from the unactivated position 1620.

As can be seen in FIG. 18, the status module 1610 is detachable from the housing 1604. The status module 1610 is secured to the housing 1604 via the mechanical coupling 1612 having reusable mechanical interlocking components such as flexible clips 1640 that cooperate with associated recesses 1642. However, any reusable mechanical interlocking arrangement known to the artisan is suitable.

In an example embodiment, when the status module 1610 is not associated with the housing interface 1606, the switch 1614 defaults to the switch first position 1624 (see FIG. 16C). In the example embodiment of FIG. 16A to 17C, once the status module 1610 is associated with (e.g., secured to) the housing 1604 at the housing interface 1606, if the movable component 1602 is in the unactivated position 1620, the position of the switch 1614 will not change. Alternately, once the status module 1610 is associated with the housing 1604 at the housing interface 1606, if the movable component 1602 is in the activated position 1622, the position of the switch 1614 will change. Likewise, once the status module 1610 is associated with the housing 1604 at the housing interface 1606, if the leading edge 1630 of the movable component 1602 is past the threshold position 1632, the position of the switch 1614 will change. As such, the status module 1610 need not be secured to the housing 1604 at the time of the activation or the simulated activation to later indicate whether the activation or the simulated activation has occurred. Instead, the status module 1610 may be secured to the housing 1604 after the activation or the simulated activation has occurred.

While not explicitly shown, the opposite is equally possible. For example, when the movable component 1602 is in the unactivated position 1620, there could be a portion of the movable component 1602 that is disposed such that is changes the position of the switch 1614 from the default switch first position 1624 to the switch second position 1626 once the switch is associated with (e.g., secured to) the housing 1604. In such an embodiment, when the switch 1614 moves toward the activated position 1622, the portion of the movable component 1602 that was under the switch 1614 could move out from thereunder upon passing the (first) threshold position, thereby causing the switch 1614 to drop from the switch second position 1626 to the switch first position 1624. The change of state of the switch 1614 could be designed to occur immediately with any movement of the movable component 1602 or after a select amount of movement of the movable component 1602.

While FIG. 16A to FIG. 17C disclose a configuration where the switch 1614 indicates whether the movable component 1602 is in the activated position 1622 or has past the threshold position 1632, the same principles can be applied to whether the movable component 1602 is in the unactivated position 1620 or has moved out of the unactivated position and past a (first) threshold.

The present inventors have created an arrangement that can provide information about one or more states of a variety of delivery devices. The arrangement does so in a way that provides great design flexibility and in a way that provides the ability to expand, all while minimizing costs and resources. Hence, this represents an improvement in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

The invention claimed is:

1. An apparatus, comprising:
at least one delivery device, each delivery device configured to be either a medicament delivery device or a medicament delivery device trainer, wherein each delivery device comprises:
a housing;
a movable component configured to dispense or to simulate dispensing a medicament; and
an unpowered electrical circuit comprising a first lead that is externally accessible, a second lead that is externally accessible, an electrical loop between the first lead and the second lead, and a switch;
wherein a change in a state of the delivery device either toggles the switch between a closed position and an open position or changes an electrical property of the switch.

2. The apparatus of claim 1, wherein the change in state toggles the switch; wherein a position of the switch is associated with a position of the movable component; and wherein the change in state comprises a select movement of the movable component.

3. The apparatus of claim 2,
wherein movement of the movable component from an undispensed position to a dispensed position dispenses or simulates dispensing of the medicament; and
wherein the select movement comprises moving the movable component from the undispensed position.

4. The apparatus of claim 2,
wherein movement of the movable component from an undispensed position to a dispensed position dispenses or simulates dispensing of the medicament; and
wherein the select movement comprises moving the movable component into the dispensed position.

5. The apparatus of claim 1, wherein the change in state toggles the switch; wherein the switch comprises a temperature responsive switch; and wherein the change in state comprises a temperature associated with the delivery device reaching or exceeding a predetermined temperature.

6. The apparatus of claim 1, wherein the change in state toggles the switch; wherein the switch comprises a temperature responsive switch; and wherein the change in state comprises a temperature associated with the delivery device warming to at least a predetermined temperature.

7. The apparatus of claim 6, wherein the predetermined temperature is a minimum temperature appropriate for dispensing the medicament.

8. The apparatus of claim 6, wherein the temperature responsive switch is configured to permanently change the state once the temperature associated with the delivery device warms to at least the predetermined temperature, and
wherein the predetermined temperature wherein the predetermined temperature is a minimum temperature appropriate for storing the medicament.

9. The apparatus of claim 1, wherein the change in state changes the electrical property of the switch; wherein the switch comprises a temperature responsive switch; wherein the state comprises a current temperature of the delivery device, and wherein the electrical property indicates a current temperature associated with the delivery device.

10. The apparatus of claim 9, wherein the electrical property comprises a resistance of the switch.

11. The apparatus of claim 1, wherein the electrical loop comprises a conductive plastic.

12. The apparatus of claim 1, wherein each delivery device further comprises:
an energy harvesting device configured to harvest energy from movement of the movable component; and
power supply leads that are externally accessible and are configured to deliver the energy harvested from the movement of the movable component.

13. The apparatus of claim 1, further comprising a status module configured:
to interface with the housing via a delivery device interface of the delivery device; and
to establish electrical communication with the unpowered electrical circuit and thereby determine the state of the delivery device.

14. The apparatus of claim 13, wherein the status module comprises two standard module interface unpowered electrical circuit leads configured to abut the first lead and the second lead when the status module standard interface interfaces with the delivery device interface.

15. The apparatus of claim 13, wherein the status module comprises two standard module interface unpowered electrical circuit leads configured to mate with the first lead and the second lead when the status module standard interface interfaces with the delivery device interface.

16. The apparatus of claim 13, wherein the status module comprises a status module standard interface configured to be releasably secured to the delivery device interface.

17. The apparatus of claim 13, wherein each delivery device further comprises an energy harvesting device configured to harvest energy from movement of the movable component and to deliver the energy harvested from the movement of the movable component to the status module when the status module interfaces with the delivery device.

18. The apparatus of claim 13, wherein the status module is configured to detect whether the switch is in the open position or the closed position and thereby determine the state of the delivery device.

19. The apparatus of claim 13, wherein the status module is configured to measure the electrical property of the switch and thereby determine the state of the delivery device.

20. The apparatus of claim 13, wherein the status module further comprises a communication device configured to communicate the state of the delivery device to a discrete reader.

21. The apparatus of claim 20, wherein the status module further comprises a power source configured to power the status module and to establish the electrical communication with the unpowered electrical circuit.

22. The apparatus of claim 20, wherein the communication device comprises a wireless communication device.

23. The apparatus of claim 22, wherein the wireless communication device comprises a Bluetooth® communication device or a Bluetooth® broadcasting device.

24. The apparatus of claim 13, further comprising a controller in data communication with the status module, configured to receive status data from the status module regarding the state of the delivery device, and configured to generate an error correction action for a user based on the status data.

25. The apparatus of claim 24, further comprising a reader in data communication with the status module and in data communication with the controller, configured to receive the status data from the status module and deliver the status data to the controller, configured to receive the error correction action from the controller, and configured to display the error correction action.

26. The apparatus of claim 1,
wherein the at least one delivery device comprises a first delivery device and a second delivery device;
wherein the apparatus further comprises a status module comprising a status module standard interface that is:
configured to interface with a first device interface comprising the first lead and the second lead of the first delivery device and to establish electrical communication with the unpowered electrical circuit and thereby determine the state of the first delivery device; and
configured to interface with a second device interface comprising the first lead and the second lead of the second delivery device and to establish electrical communication with the unpowered electrical circuit of the second delivery device and thereby determine the state of the second delivery device; and
wherein a configuration of the first delivery device and a configuration of the second delivery device are different from each other.

27. The apparatus of claim 26, wherein the status module standard interface is configured to be releasable secured to the first device interface and to be releasable secured to the second device interface.

28. The apparatus of claim 26,
wherein the status module standard interface comprises a plurality of standard module interface leads comprising two standard module interface unpowered electrical circuit leads;
wherein the first device interface comprises the first lead and the second lead of the first delivery device;
wherein the second device interface comprises the first lead and the second lead of the second delivery device; and
wherein the first lead and the second lead of the first delivery device and the first lead and the second lead of the second delivery device are configured to connect to the two standard module interface unpowered electrical circuit leads.

29. The apparatus of claim 26,
wherein the status module standard interface comprises a plurality of standard module interface leads;
wherein the first device interface comprises a plurality of first device interface leads comprising the first lead and the second lead of the first delivery device;
wherein the second device interface comprises a plurality of second device interface leads comprising the first lead and the second lead of the second delivery device;
wherein the plurality of first device interface leads connect to a first set of standard module interface leads of the plurality of standard module interface leads;
wherein the plurality of second device interface leads connect to a second set of standard module interface leads of the plurality of standard module interface leads; and
wherein the first set of standard module interface leads is different than the second set of standard module interface leads.

30. The apparatus of claim 29,
wherein the plurality of standard module interface leads comprises two standard module interface unpowered electrical circuit leads; and
wherein the first lead and the second lead of the first delivery device and the first lead and the second lead of the second delivery device are configured to connect to the two standard module interface unpowered electrical circuit leads.

31. The apparatus of claim 1, wherein the at least one delivery device comprises the medicament delivery device trainer.

32. An apparatus, comprising:
a delivery device, comprising:
a housing;
a movable component configured to dispense or to simulate dispensing a medicament; and
an unpowered electrical circuit comprising a first lead, a second lead, an electrical loop, and a switch between the first lead and the second lead; wherein a change in a state of the delivery device toggles the switch between a closed position and an open position;
a delivery device interface comprising the first lead and the second lead; wherein the delivery device interface is configured to interface with a standard module interface of a status module.

33. The apparatus of claim 32, wherein a position of the switch is associated with a position of the movable component; and wherein the change in state comprises a select movement of the movable component.

34. The apparatus of claim 33, wherein the switch comprises a conductive material, and wherein the select movement moves the conductive material into simultaneous contact with the first lead and the second lead.

35. The apparatus of claim 34, wherein the movable component dispenses or to simulates dispensing the medicament by moving through a dispensing stroke, and wherein the delivery device is configured to move the conductive material into simultaneous contact with the first lead and the second lead as the movable component reaches an end of the dispensing stroke.

36. The apparatus of claim 35, wherein the movable component is composed of the conductive material.

37. The apparatus of claim 35, wherein the conductive material is secured to the movable component.

38. The apparatus of claim 32,
wherein the delivery device further comprises a second unpowered electrical circuit comprising a third lead and a fourth lead; and
wherein the delivery device interface comprises the third lead and the fourth lead.

39. The apparatus of claim 32, further comprising the status module comprising a status module standard interface that is configured to establish electrical communication with the first lead and the second lead and thereby determine the state of the delivery device.

40. The apparatus of claim 39, wherein the status module standard interface comprises two leads that abut the first lead and the second lead when the status module standard interface interfaces with the delivery device interface.

41. The apparatus of claim 39, wherein the status module standard interface comprises two leads that mate with the first lead and the second lead when the status module standard interface interfaces with the delivery device interface.

42. The apparatus of claim 39, wherein the status module standard interface is configured to be releasably secured to the delivery device interface when the status module standard interface interfaces with the delivery device interface.

43. The apparatus of claim 39, wherein the status module is configured to detect whether the switch is in the open position or the closed position to thereby determine the state of the delivery device.

44. The apparatus of claim 43, wherein the status module further comprises a communication device configured to communicate data from the delivery device to a discrete reader.

45. The apparatus of claim 44, wherein the status module further comprises a power source configured to power the status module and to establish the electrical communication with the unpowered electrical circuit.

46. The apparatus of claim 45, wherein the communication device comprises a wireless communication device.

47. An apparatus, comprising:
a delivery device that is configured to be either a medicament delivery device or a medicament delivery device trainer and that comprises: a housing; a housing interface; and a movable component configured to dispense or to simulate dispensing a medicament; and
a status module that is configured to cooperate with the housing interface and that comprises a switch;
wherein a position of the switch depends on a position of the movable component; and
wherein the status module is configured to indicate the position of the switch.

48. The apparatus of claim 47,
wherein the delivery device is configured such that initiation of a dispensing operation or a simulated dispensing operation occurs when the movable component moves past a first threshold position; and
wherein when the status module is associated with the housing interface the switch changes position when the movable component moves beyond the first threshold position.

49. The apparatus of claim 47, wherein the delivery device is configured such that initiation of a dispensing operation or a simulated dispensing operation occurs when the movable component moves past a first threshold position;
wherein when the status module is not associated with the housing interface the switch defaults to a first position; and
wherein upon associating the status module with the housing interface, the switch does not change position.

50. The apparatus of claim 47, wherein the delivery device is configured such that initiation of a dispensing operation or a simulated dispensing operation occurs when the movable component moves past a first threshold position;
wherein when the status module is not associated with the housing interface the switch defaults to a switch first position; and
wherein upon associating the status module with the housing interface, the switch changes position.

51. The apparatus of claim 47, wherein the delivery device is configured such that a sufficient completion of a dispensing operation or a simulated dispensing operation occurs when the movable component crosses a second threshold position; and
wherein when the status module is associated with the housing interface the switch changes position when the movable component reaches the second threshold position.

52. The apparatus of claim 47, wherein the delivery device is configured such that a sufficient completion of a dispensing operation or a simulated dispensing operation occurs when the movable component reaches a second threshold position; and
wherein when the status module is not associated with the housing interface the switch defaults to a first position; and
wherein upon associating the status module with the housing interface, the switch does not change position.

53. The apparatus of claim 47, wherein the delivery device is configured such that a sufficient completion of a dispensing operation or a simulated dispensing operation occurs when the movable component reaches a second threshold position; and
wherein when the status module is not associated with the housing interface the switch defaults to a first position; and
wherein upon associating the status module with the housing interface, the switch changes position.

54. The apparatus of claim 47, wherein the status module further comprises a communication device configured to wirelessly communicate the position of the switch to a discrete reader.

55. The apparatus of claim 54, further comprising a controller in data communication with the status module, configured to receive status data from the status module regarding a state of the delivery device, and configured to generate an error correction action for a user based on the status data.

56. The apparatus of claim 55, further comprising the discrete reader in data communication with the status module and in data communication with the controller, configured to receive the status data from the status module and deliver the status data to the controller, configured to receive the error correction action from the controller, and configured to display the error correction action.

57. The apparatus of claim 54, further comprising the discrete reader in data communication with the status module and configured to provide instructions to a user.

58. The apparatus of claim 54, further comprising the discrete reader in data communication with the status module and configured to track adherence to a treatment regimen.

59. The apparatus of claim 47, wherein the movable component comprises a needle shield or a plunger.

60. The apparatus of claim 47, further comprising a mechanical coupling configured to associate the status module with the housing interface via a reusable mechanical interlock.
